(19) United States
(12) Patent Application Publication
(10) Pub. No.: US 2026/0318855 A1
(43) Pub. Date: Oct. 15, 2026

(54) COPPER DETECTION TECHNIQUES USING NEUTRONS AND GAMMA-RAYS

(71) Applicants: Shikha Prasad, College Station, TX (US); Vijay Kumar Prasad, College Station, TX (US)

(72) Inventors: Shikha Prasad, College Station, TX (US); Vijay Kumar Prasad, College Station, TX (US)

(21) Appl. No.: 19/320,602

(22) Filed: Sep. 5, 2025

(51) Int. Cl. G01N 23/222; G01N 15/00; G01N 15/06; G01N 23/02; G01N 33/20

(52) U.S. Cl. CPC G01N 23/222; G01N 15/06; G01N 23/025; G01N 33/20; G01N 2015/0053; G01N 2223/0745; G01N 2223/635

(57) ABSTRACT

A system includes one or more radiation sources configured to emit neutrons. The system also includes one or more gamma-ray detectors configured to detect gamma-rays following neutron activation of copper. Further, the system includes a control system comprising one or more processors. The control system is configured to determine a copper concentration based on the gamma-rays. Further, the control system is configured to generate a copper extraction output based on the copper concentration.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

Copper is vital to the world economy because it is a foundational material for modern infrastructure and technology. Copper is the most widely used electrical conductor, making it essential for everything that uses electricity. Copper is a key element in electric vehicles (EV), renewable energy, grid modernization and energy storage. Renewable energy technologies such as solar and wind energy are significantly more copper-intensive than traditional ones. With increasing need for reduction in greenhouse gases (GHG) and demand for fuel cell energy storage for EV and hybrid vehicles (HEV); renewable, but non-transportable, energy sources such as solar and wind energy for various applications including smart-grids grows; demand for technologies to store energy generated using such sources also grows, leading to an increasing use of copper.

According to the United States Geological Survey, global reserves of copper total 980 million tons (metric) of copper content, with Chile, Peru, Australia, Russia and Democratic Republic of the Congo, accounting for over 50% of global reserves. The International Energy Agency's Global Critical Minerals Outlook 2025 warns that without a massive increase in investment in new copper mining projects, the world could face a 30% supply shortfall by 2035 under current government plans worldwide. This deficit would be even larger under more ambitious climate and energy goals.

The major projected supply-demand imbalance is foreseen in the future due to many drivers. The global energy transition is the primary driver of the demand surge. Electric vehicles, renewable energy infrastructure (wind and solar), and grid modernization are all extremely copper-intensive. In addition, the massive build-out of data centers for AI is emerging as a new and powerful demand driver. The quality of copper ore from existing mines is steadily decreasing. This means that miners have to process more rock to get the same amount of copper, which increases costs and environmental impact. There has been a lack of new, high-grade copper deposits in recent years. The time from discovery to production for a new copper mine is now incredibly long, often taking 15-20 years. Geopolitical and Social Risks: Major copper-producing regions, particularly in Latin America, face political instability, regulatory challenges, and social opposition to new mining projects. This can lead to delays or the outright cancellation of projects. Accordingly, it is recognized that it is desirable to improve techniques related to copper production.

### SUMMARY

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below.

In certain embodiments, a system includes one or more radiation sources that emit neutrons. The system also includes one or more gamma-ray detectors that detect gamma-rays. Further, the system includes a control system having one or more processors. The control system determines a copper concentration based on the gamma-rays generated from neutron activation of copper. Further, the control system generates a copper extraction output based on the copper concentration.

In certain embodiments, a method includes providing a copper-containing sample. The method also includes emitting neutrons into the copper-containing sample. Further, the method includes detecting gamma-rays subsequent to emitting the neutrons. The gamma-rays correspond to neutron activation associated with copper in the copper-containing sample. Neutron activation of copper turns non-radioactive copper into other radioactive material which then emit gamma-rays. Further still, the method includes determining a copper ion concentration based on the emitted gamma-rays. Even further, the method includes adjusting one or more operations of a copper extraction system based on the copper ion concentration.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic diagram of a copper detection system, in accordance with an aspect of the present disclosure;

FIG. 2 is a schematic diagram of a downhole tool that includes an example of the copper detection system of FIG. 1, in accordance with an aspect of the present disclosure; and

FIG. 3 illustrates a flow chart of a method for determining operational adjustments using the copper detection system of FIG. 1 that includes a neutron source, in accordance with an aspect of the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are used in an open-ended fashion, and thus should be interpreted to mean “including, but not limited to.” Also, any use of any form of the terms “connect,” “engage,” “couple,” “attach,” or any other term describing an interaction between elements is intended to mean either an indirect or a direct interaction between the elements described. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. The use of “top,” “bottom,” “above,” “below,” and variations of these terms is made for convenience but does not require any particular orientation of the components. As referred to here, “about” or “approximately” a value refers to ±0.5%, ±1%, ±2%, ±5%, ±10%, or ±20%.

Determination of the presence of copper in mineral ore extraction is a challenge. Further, certain techniques for quantification of copper concentration and mass in brine is also complex. Accordingly, it is presently recognized that it is advantageous to obtain an indication of the amount of copper that could be extracted from a medium at a certain location, for instance, to take a decision on exploitation and return on investment for such locations.

Accordingly, this disclosure relates to techniques for detecting, measuring, or otherwise obtaining a concentration of copper using nuclear measurements that may inform certain mineral extraction decisions (e.g., copper extraction decisions), such as determining where to extract a copper-containing brine. Certain aspects of this disclosure relate to using a neutron source to detect gamma-rays (y) indicative of a copper concentration. It is presently recognized that it may be advantageous to utilize the relatively large cross-section (e.g., about 10 to 100 barn) for thermal neutron capture of the copper-63 and copper-65 isotopes, in combination of, or, separately to detect a copper concentration. Copper-63 (Cu-63) a stable isotope of copper having a kernel with 63 particles-29 protons and 34 neutrons, which has an abundance of approximately 69.2% of natural copper. Copper-65 (Cu-65 ) a stable isotope of copper having a kernel with 63 particles-29 protons and 36 neutrons, which has an abundance of approximately 69.2% of natural copper.

Some source neutrons that interact with Cu-63 may lead to the formation of copper-64 which is unstable and would decay with a half-life of 12.7 hours into nickel-64 (Ni-64 ) and a gamma-ray of energy 1,346 keV, as shown below:

⁶³Cu+n→⁶⁴Cu→⁶⁴Ni+γ (1,346 Kev) (1)

Some source neutrons that interact with Cu-65 may lead to the formation of copper-66 which is unstable and would decay with a half-life of 5.1 minutes into zinc-66 (Zn-66 ) and a gamma-ray of energy 1,039 keV, as shown below:

⁶⁵Cu+n→⁶⁶Cu→⁶⁶Zn+γ (1,039 keV) (2)

Accordingly, the gamma-rays may be detected using suitable detectors, such as, scintillation detectors with photomultiplier (PM), semiconductor detectors, and other detectors capable of detecting gamma-rays.

In general, the techniques may include a copper detection apparatus having a radiation source and one or more detectors arranged in suitable positions to obtain a transmission or absorption measurement in solids and/or liquids that contain copper. As described above, the radiation source may be a neutron source and the one or more detectors may be a gamma-ray detector. As described in more detail herein, in some embodiments, the radiation source may be may be a radioactive neutron source or an electronic neutron generator. In any case, the disclosed techniques may improve the accuracy of measurements for a relatively broad range of copper concentrations (e.g., between approximately 1 ppm to 2,000 ppm, 1 ppm to 10,000 ppm, 1 ppm and 100,000 ppm, or 1 ppm to saturation) in a relatively low volume. Although the disclosed embodiments are described as being implemented in a downhole tool, it should be noted that such techniques may also be implemented on the surface. In any case, the disclosed techniques provide techniques for determining copper concentration, which may facilitate mineral extraction related decisions.

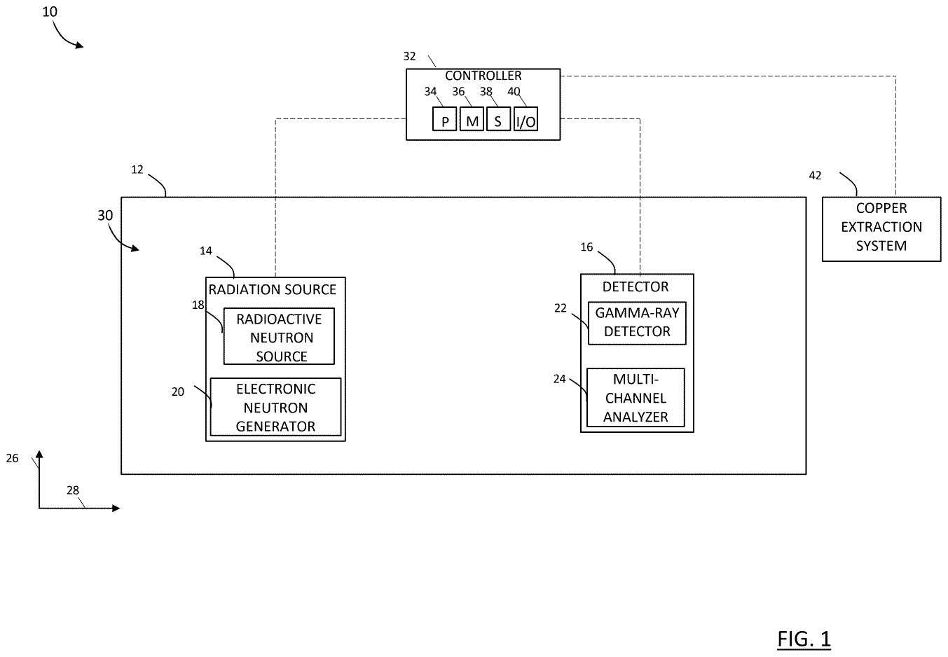

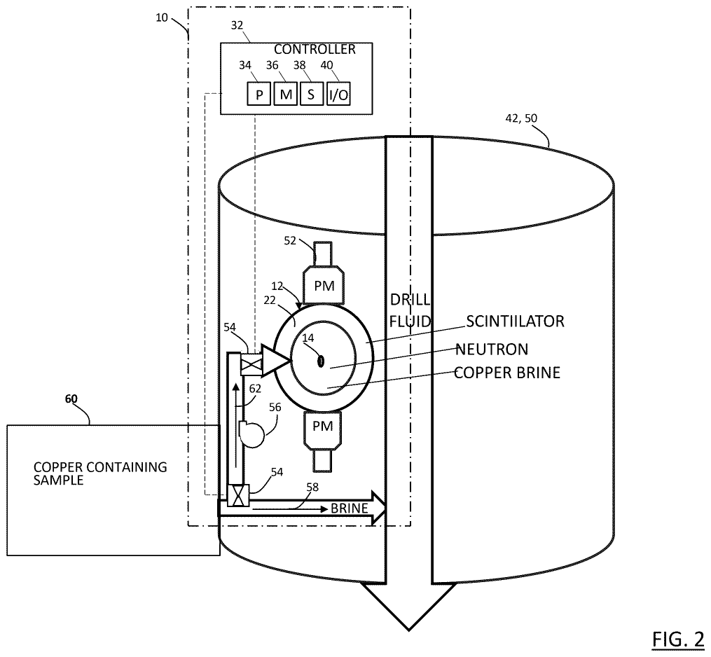

With the foregoing in mind, FIG. 1 shows an example of a copper detection system 10. As shown, the copper detection system 10 includes a vessel 12 holding a radiation source 14 and one or more detectors 16. In some embodiments, the copper detection system 10 may be implemented in a downhole tool. In some embodiments, the copper detection system 10 may be used in a fluid system that is uphole or on a surface. As described in more detail in FIG. 2, the vessel 12 may include flow control components, such as valves and pumps, that direct a fluid flow from a source (e.g., a subterranean reservoir) into the vessel 12. For example, the vessel 12 may include flow control components, such as fluid sampling modules that extract fluids from a subterranean formation. In some embodiments, the vessel 12 may include sample introduction components, such as conveyor belts or robotic arms that are capable of introducing a solid sample into the vessel 12.

As described herein, the radiation source 14 may include a radioactive neutron source 18 or an electronic neutron generator 20. As described in more detail herein, in embodiments when the radiation source 14 includes a radioactive neutron source 18 and/or electronic neutron generator 20, the detector 16 may include a gamma-ray detector 22. In some embodiments, the detector 16 may also include a multi-channel analyzer 24 to count the gamma-ray in particular channel based on its energy deposited in the detector 22. In some embodiments, the radiation source 14 may include one or more radioactive neutron sources 18 or one or more electronic neutron generators 20. In some embodiments, the detector 16 may include one or more gamma-ray detectors 22 and/or one or more multi-channel analyzers 24.

As shown, the radiation source 14 and the detector 16 are arranged in different positions within the vessel 12 along the lateral axis 26 and the longitudinal axis 28 with the volume 30 of the vessel 12. It should be noted that the arrangement of the radiation source 14 and the detector 16 shown in FIG. 1 are meant to be non-limiting. In some embodiments, the detector 16 may arranged around one or more walls or a perimeter of the vessel 12 such that the detector 16 substantially surrounds the radiation source 14 (e.g., surrounds about, about 3%, 6%, about 10%, about 20%, about 30%, about 40%, about 50%, about 75%, about 90%, or about 95% of the area around the radiation source 14). For example, the detector 16 may be arranged in one or more radial positions about the radiation source 14. As such, the vessel 12 may have an area of about 50 cm² or less, about 100 cm² or less, about 250 cm² or less, about 500 cm² or less, about 1,000 cm² or less, about 2,000 cm² or less and so on.

In general operation, the radiation source 14 may emit a particle, such as a neutron, into the volume 30. The detectors 16 may detect a gamma-ray when the emitted particle is a neutron. Accordingly, the detectors 16 may obtain measurements indicating a count of measured events (e.g., gamma-rays detected by the detectors 16). The measurements obtained by the one or more detectors 16 and/or operation of the radiation sources 14 may be received by a controller 32 to adjust, modify, or otherwise control operations of the copper detection system 10 and/or copper extraction system 42. As illustrated, the controller 32 includes a processor 34, memory 36, storage 38, and an input/output (I/O) port 40.

The processor 34 (e.g., processing circuitry) may be any type of computer processor or microprocessor capable of executing computer-executable code. While only one processor 34 is shown, in some embodiments, the controller 32 may include multiple processors 34 that may perform the operations described herein. The memory 36 (e.g., memory circuitry) and the storage 38 may be any suitable articles of manufacture that can serve as media to store processor-executable code, data, or the like. These articles of manufacture may represent computer-readable media (e.g., any suitable form of memory or storage) that may store the processor-executable code used by the one or more processors of the processor 34 to perform the presently disclosed techniques. As used herein, applications may include any suitable computer software or program that may be installed onto the controller 32 and executed by the processor 34. The memory 36 and the storage 38 may represent non-transitory computer-readable media (e.g., any suitable form of memory or storage) that may store the processor-executable code used by the processor 34 to perform various techniques described herein. It should be noted that non-transitory merely indicates that the media is tangible and not a signal. The I/O ports 40 may be interfaces that may couple to other peripheral components, such as input devices (e.g., keyboard, mouse), detectors, input/output (I/O) modules, and the like.

In any case, the controller 32 may be control operation of a copper extraction system 42 based on the measurements obtained by the detector(s) 16. In general, the copper extraction system 42 may include a sampling system, a mineral extraction system (e.g., that obtains a copper-containing sample, such as a solid or fluid), and so on. For example, the copper extraction system 42 may be a downhole tool.

As described herein, the copper detection system 10 may be implemented in a downhole tool. To illustrate this, FIG. 2 shows a schematic diagram of a downhole tool 50 (e.g., a copper extraction system 42) that includes the copper detection system 10. In the illustrated embodiment, the copper detection system 10 includes a radiation source 14, a gamma-ray detector 22, and a photomultiplier (PM) 52. Further, the copper detection system 10 includes the controller 32 (e.g., having the processor 34, the memory 36, the storage 38, and the I/O ports 40). As shown, the copper detection system 10 also includes one or more valves 54 and one or more pumps 56 that are communicatively coupled to the controller 32, as discussed in more detail below. In some embodiments, the downhole tool 50 may include one or more shields/moderators in the vicinity of the radiation source 14 configured to thermalize the neutrons emitted before the neutrons reach the medium (e.g., that potentially includes copper). In some embodiments, the downhole tool 50 may be conveyed by a conveyance line into a geological formation.

In operation, a fluid flow 58 (e.g., sample flow) from the copper-containing sample fluid 60 (e.g., a subterranean reservoir, subsurface reservoir) may be directed into the downhole tool 50 via a fluid sampling module of the downhole tool 50. To obtain measurements of the copper concentration in the copper-containing fluid 60, the controller 32 may adjust (e.g., open or close) operation of the one or more valves 54 and/or the one or more pumps 56 to cause a sample fluid flow 62 to flow (e.g., along a flow path within a conduit) into the vessel 12 that includes the radiation source 14 and the gamma-ray detector 22. In some embodiments, the controller 32 may open the valves 54, such that the flow rate of the sample fluid flow 62 is within a threshold range (e.g., 10% or less, 20% or less, 30% or less, 40% or less, or 50% or less than the flow of the fluid flow 58). In some embodiments, the vessel 12 may be disposed along the fluid flow 58 as compared to in a bypass or otherwise a separate flow arrangement. Although only one vessel 12 is shown, it should be noted that the copper extraction system 42 may include any suitable number of vessels 12, such as 2, 3, 4, 5, or more than 5. Although the vessel 12 is shown as being cylindrical or otherwise having a circular cross section, it should be noted that the vessel 12 may be any suitable shape, such as rectangular. In some embodiments, the dimensions of the vessel 12 (e.g., the length, the width, or the diameter) may be between about 20 to 30 cm. For example, the vessel 12 may have a diameter of about 20, about 21, about 22, about 23, about 24, about 25 cm, or so on. In some embodiments, the diameter may be between about 5 to 40 cm, 5 to 35 cm, 10 to 30 cm, or 15 to 30 cm. It should be noted that certain descriptions may also apply to a vessel 12 that receives a copper-containing sample that is a solid. For example, the controller 32 may control a robotic arm that directs a solid sample into the vessel 12.

In any case, the copper in the sample fluid flow 62 may interact with the neutrons. Put differently, the neutrons from the radiation source 14 may activate the copper in the brine, thereby producing gamma-rays. The gamma-ray produced by copper neutron activation may be detected by the gamma-ray detector 22. For example, when the gamma-ray detector 22 is a scintillator, the scintillation material of the scintillator may produce light due to the interaction with the scintillation material and the gamma-rays. The PM 52 detects the light emitted by the scintillator, which is then counted and converted into a pulse height spectrum by the processor 34.

As described herein by Eq. (1) and Eq. (2) the gamma-ray detector 22 may be any suitable gamma-ray detector capable of detecting gamma-rays having any energy less than or equal to 1,500 keV, such that the 1,346 keV and 1,039 keV gamma-rays can be sufficiently detected even with poor energy-resolution. In some embodiments, the gamma-ray detector 22 may be a scintillator that include two or more different scintillation materials. It is presently recognized that it may be advantageous to use such scintillation materials because they may be easy to mold into different shapes.

In some embodiments, the one or more valves 54 may be one or more one-way valves. As such, the valve 54 may allow the sample-flow 62 to flow into the vessel 12 until the vessel 12 is filled. In some embodiment, the processor 34 may periodically cause the electronic neutron generator 20 to activate.

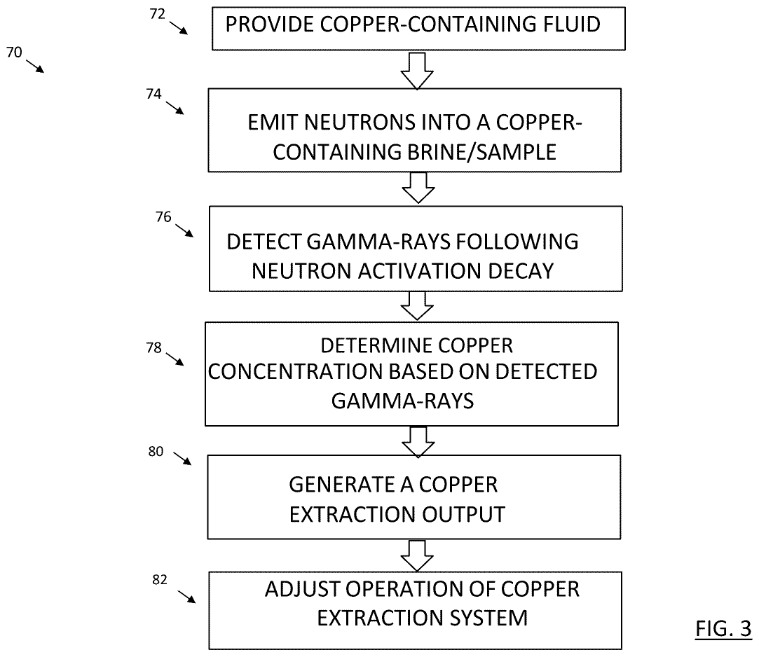

FIG. 3 illustrates a flowchart of a process 70 for monitoring a copper concentration (e.g., a copper-ion concentration) within a sample (e.g., a copper-containing brine stream) from a copper-containing fluid 60. One or more blocks of the process 70 may be implemented by a processor 34 of the controller 32 for modifying operation of the copper detection system 10 and/or the copper extraction system 42 based on a measured copper concentration by the copper detection system 10. Although certain steps of the process 70 are described as being performed by the processor 34, it should be noted that any suitable processing device may be capable of performing the process 70. In some embodiments, one or more of the blocks may be omitted or performed in a different order than shown.

At block 72, the processor 34 provides a copper-containing sample (e.g., a copper-containing fluid, a copper-containing brine, a copper-containing solid) to a copper detection system 10. For example, controller 32 may actuate one or more valves 54 to direct a sample fluid flow 62 and/or the fluid flow 58 into the vessel 12 that includes the radiation source 14 and the one or more detectors 22. It should be noted that in some embodiments, the copper-containing sample may be a copper-containing solid dispersed within a liquid (e.g., a slurry). In embodiments where the copper-containing sample is a solid, the controller 32 may actuate, for example, a robotic arm to introduce a solid copper-containing sample. Additionally or alternatively, if the copper detection system 10 is implemented on the surface, the controller 32 may send a control signal that causes the copper detection system 10 to determine a copper ion concentration within a sample chamber (e.g., a vessel) and/or a direct measurement. For example, the copper detection system 10 may be configured to directly measure the formation (e.g., the walls of a subterranean formation as opposed to measuring a sample within the downhole tool). In some embodiments, providing the copper-containing fluid may include activating one or more fluid sample modules of a downhole tool. For example, a downhole tool 50 may be lowered into a wellbore to a particular depth. Once at the depth, the processor 34 may send a control signal that causes one or more fluid sample modules to activate, thereby providing the sample fluid flow 62 and/or the fluid flow 58 into the vessel 12.

At block 74, the processor 34 causes the radiation source 14 to emit neutrons into the copper-containing sample within the vessel 12. For example, the controller 32 may activate one or more electronic neutron generators 20 that cause neutrons to be emitted into the volume 30 of the vessel 12. At block 76, the gamma-ray detector 22 detects gamma-rays corresponding to neutron activation decay. As described herein, it is presently recognized that copper-63 and copper-65 present in the copper-containing fluid may have a relatively high capture cross section for interacting with thermal neutrons. In turn, the neutrons may be captured by copper to release gamma-rays. In some embodiments, the processor 34 may activate other types of detection apparatus within the vessel 12. For example, a neutron measurement may be combined with one or more analytical measurements to determine a concentration of the copper within the sample fluid flow 62.

At block 78, the processor 34 measures or determines a copper ion concentration based on the detected gamma-rays. For example, in some embodiments, the neutron measurement is used to determine that a copper concentration is within a window/threshold range or above a threshold concentration (e.g., a minimum copper concentration) in the vessel 12. In some embodiments, the processor 34 may determine a flow rate and/or volume of the sample fluid flow 62 and/or the fluid flow 58. As such, the processor 34 may utilize the flow rate and/or volume to determine an amount of copper that has entered the downhole tool 50 via the fluid flow 58. In some embodiments, the processor 34 may obtain in situ measurements of the copper ion concentration. That is, the processor 34 may record one or more multiple measurements of a copper concentration over a time period to provide “on the fly” measurements of the copper concentration. Such measurements may aid operators in making more rapid decisions based on the concentration of copper. For example, the processor 34 may monitor a trend of the measured or determined copper ion concentration, such as a rate of change of the copper ion concentration. If the copper ion concentration exceeds a threshold, the processor 34 may generate the copper extraction output mentioned below such that it indicates the trend. In some embodiments, the processor 34 make take certain control actions based on the rate of change being above, below, or within a threshold (e.g., a copper ion concentration rate threshold). For example, if the rate of change of concentration exceeds a threshold, the processor 34 may adjust the position of valves 54 to increase or toggle the fluid flow 58 within the downhole tool 50 (e.g., through a mud channel within the downhole tool 50). In some embodiments, the processor 34 may cause the copper extraction system 42 to operate and extract fluid and/or solid from copper containing geological formation and/or copper-containing fluid 60. In this way, the processor 34 may determine the concentration of copper over a predetermined time period or otherwise as the measurements are being recorded.

At block 80, the processor 34 may determine or generate a copper extraction output based on the copper concentration. In general, the copper extraction output may include an alert or control signal that causes a modification to the operation of the copper extraction system 42 (e.g., adjust a position of a valve 54 or speed of a pump 56). As such, the copper extraction output may aid an operator or user in determining where there is a sufficient amount of copper for extraction. In some embodiments, the processor 34 may detect gamma-rays using the gamma-ray detectors and returning an indicator relative to a gamma-ray count (e.g., a number over a time period or a number per unit time (count rate)). As such, the processor 34 may relate the gamma-ray count (and thus copper activation) to an indication of the amount of copper at location. This may be provided within the tool via the processor 34 or at control system at the surface based on communication of the gamma-ray count by the tool. For example, the processor 34 may transmit an alert to a separate computing device (e.g., a computing device on the surface as opposed to one downhole) if the gamma-ray count falls within a counting count. In some embodiments, the processor 34 may still transmit an indication (e.g., a visual indication or other alert) that indicates the gamma-ray count and/or whether the gamma-ray count is within a window corresponding to the full energy peaks or other characteristic parts of the energy spectra (e.g., Compton continuum) of the gamma-rays in Eq. (1) or Eq. (2), or other data indicative of a copper ion concentration in a sample.

At block 82, the processor 124 adjusts operation of the copper detection system 10 and/or copper extraction system 42 based on the copper extraction output. For example, the processor 34 may output a control signal that causes an actuator to actuate, thereby modifying a position of valve 54 or other mechanical component of the copper extraction system 42. For example, the processor 34 may determine that the concentration of copper in the fluid flow 58 (e.g., a first fluid flow) exceeds a concentration threshold indicating it may be advantageous to continue or increase the flow rate of the fluid flow 58 into the copper extraction system 42. As such, the copper extraction output may be a control signal that reduces the speed of a pump 56 -, adjusts a position of a valve 54 (e.g., opens a valve), causes a display of a computing device to display an alert indicating the concentration of the copper in the copper-containing fluid 60 or amount of copper that has entered the copper extraction system 42.

Technical effects of the present disclosure include techniques for determining copper concentration of solids or liquids while downhole. For example, the techniques of the present disclosure may be used for in situ detection of copper, as opposed to measuring the copper concentration on the surface. This may allow operators to more quickly develop copper extraction decisions. For example, the disclosed techniques may be utilized to obtain copper concentration measurements at multiple depths during a downhole operation (e.g., logging-while-drilling). As such, the measurements by the disclosed copper detection system may be used to control a downhole tool.

In one embodiment, the method was tested in a controlled environment on the surface to validate one or more blocks of the process 70. A sample of copper with a mass of about 96 g containing both copper-63 and copper-65 isotopes was irradiated with a radioactive neutron source for over 28 hours. The resulting sample measured with a high purity germanium gamma-ray detector. The gamma-ray measurement was performed for approximately 26 minutes following the irradiation and yielded clearly identifiable full-energy peaks at 1,039 keV (with over 500 counts in the peak) and at 1,346 keV (with over 300 counts in the peak), presented in Eq. (1) and Eq. (2). These results are provided by way of example only and should not be considered limiting. It is contemplated that variations in processing conditions, and/or measurement configurations may yield different values while still remaining within the scope of the present invention.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112(f). However, for any claims containing elements designated in any other manner, it is intended that such elements are not to be interpreted under 35 U.S.C. 112(f).

1. A system, comprising:
one or more radiation sources configured to emit neutrons;
one or more gamma-ray detectors configured to detect gamma-rays; and
a control system comprising one or more processors, wherein the control system is configured to:
determine a copper concentration based on the gamma-rays; and
generate a copper extraction output based on the copper concentration.

2. The system of claim 1, comprising a downhole tool that includes the one or more neutron sources (including electronic neutron generators) and the one or more gamma-ray detectors.

3. The system of claim 1, comprising one or more valves and a vessel, wherein the control system is configured to adjust a position of the valves to cause a sample fluid flow to enter the vessel.

4. The system of claim 1, further comprising a vessel configured to hold a copper-containing sample, wherein the one or more radiation sources are configured to emit the neutrons towards the vessel.

6. The system of claim 1, wherein the one or more gamma-ray detectors comprise a scintillator and a photomultiplier.

7. The system of claim 1, wherein the copper extraction output is configured to adjust operation of a copper extraction system.

8. The system of claim 1, further comprising a vessel, wherein the copper-containing sample comprises a copper-containing fluid, and wherein the vessel comprises one or more one-way valves that provide a flow path of the fluid into the vessel.

9. The system of claim 1, wherein the copper extraction output comprises indicates an in situ measurement of the copper concentration.

10. A method, comprising:
providing a copper-containing sample;
emitting neutrons into the copper-containing sample;
detecting gamma-rays subsequent to emitting the neutrons, wherein the gamma-rays correspond to the copper concentration in the copper-containing sample;
determining a copper ion concentration based on the detected gamma-rays; and
adjusting one or more operations of a mineral extraction system based on the copper ion concentration.

11. The method of claim 10, wherein the copper-containing sample comprises a sample fluid flow from a subsurface reservoir.

12. The method of claim 11, wherein providing the copper-containing sample comprises activating a valve to direct the sample fluid flow into a vessel.

13. The method of claim 11, wherein adjusting the one or more operations of the mineral extraction system comprises actuating a valve to direct the sample flow into a mud channel.

14. The method of claim 10, further comprising performing a background signal subtraction to remove background noise; and determining the copper ion concentration based on the background signal subtraction.

15. The method of claim 10, wherein emitting the neutrons comprises activating an electronic neutron source disposed within a downhole tool.

16. The method of claim 10, where in determining the copper ion concentration comprises receiving a count of the gamma-rays using a photomultiplier.
